(19) United States
(12) Patent Application Publication
(10) Pub. No.: US 2026/0335115 A1
(43) Pub. Date: Oct. 29, 2026

(54) LEG BRACE ASSEMBLY AND METHOD THEREFOR

(71) Applicant: Gary Meyer, Elgin, TX (US)

(72) Inventor: Gary Meyer, Elgin, TX (US)

(21) Appl. No.: 19/193,333

(22) Filed: Apr. 29, 2025

(51) Int. Cl. A61F 5/01

(52) U.S. Cl. CPC A61F 5/0123

(57) ABSTRACT

A leg brace assembly includes a leg brace, a strap, a first coupler element, and a second coupler element. The strap is designed to be worn in a loop around a torso and over a shoulder opposite a leg on which the leg brace is worn. The first coupler element is attached to the strap. The second coupler element is attached to the leg brace. The second coupler element is coupled to the first coupler element to detachably couple the strap to the leg brace, such that the first coupler element and the second coupler element extend along a hip of the leg to support the leg brace on the strap in a suspended manner to minimize sliding movement of the leg brace down the leg.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to orthopedic and rehabilitative leg braces and more particularly pertains to a new leg brace assembly for minimizing sliding movement of the leg brace down the leg. Leg braces are worn for support and stabilization of injured or medically-repaired legs, particularly after knee injuries or knee surgery. Several straps of elastic or hook-and-loop material hold the leg brace on the leg, but unfortunately during wear, the leg brace can become loose and slide down the leg due to a combination of such factors as gravity, sweat, and vibration during walking or running. The new leg brace assembly provides a shoulder strap to support the leg brace and prevent or minimize such sliding movement.

### (2) Description of Related Art including information disclosed under 37 CFR 1.97 and 1.98

The prior art relates to orthopedic and rehabilitative leg braces. The prior art, as best understood, does not disclose a leg brace assembly that includes a leg brace, a strap, a first coupler element, and a second coupler element, wherein the strap is designed to be worn in a loop around a torso and over a shoulder opposite a leg on which the leg brace is worn, and the first coupler element and the second coupler element extend along a hip of the leg to support the leg brace on the strap in a suspended manner to minimize sliding movement of the leg brace down the leg.

### BRIEF SUMMARY OF THE INVENTION

An embodiment of the disclosure meets the needs presented above in a leg brace assembly generally comprising a leg brace, a strap, a first coupler element, and a second coupler element. The strap is designed to be worn in a loop around a torso and over a shoulder opposite a leg on which the leg brace is worn. The first coupler element is attached to the strap. The second coupler element is attached to the leg brace. The second coupler element is coupled to the first coupler element to detachably couple the strap to the leg brace, such that the first coupler element and the second coupler element extend along a hip of the leg to support the leg brace on the strap in a suspended manner to minimize sliding movement of the leg brace down the leg ..

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a leg brace assembly according to an embodiment of the disclosure in use.

FIG. 2 is a rear view of an embodiment of the disclosure in use.

FIG. 3 is a side view of an embodiment of the disclosure.

FIG. 4 is a front view of an embodiment of the disclosure in use.

### DETAILED DESCRIPTION OF THE INVENTION

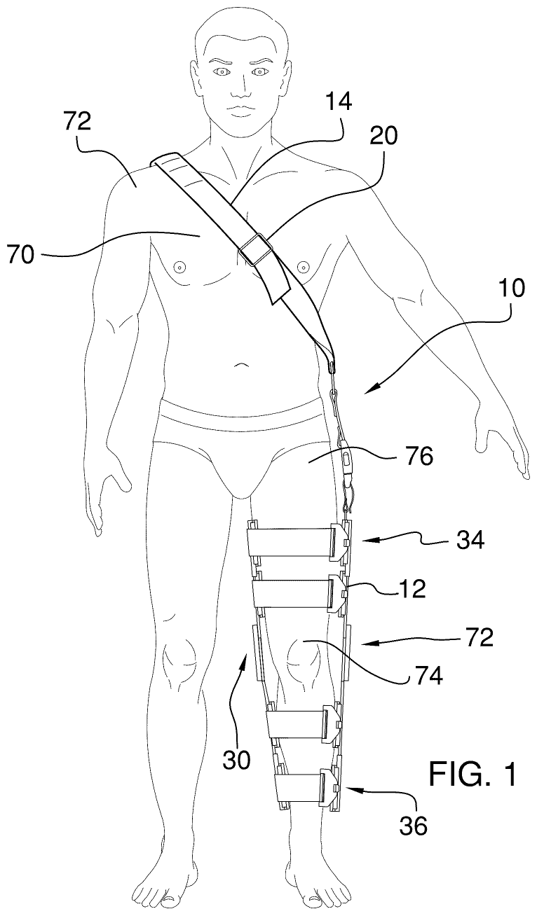

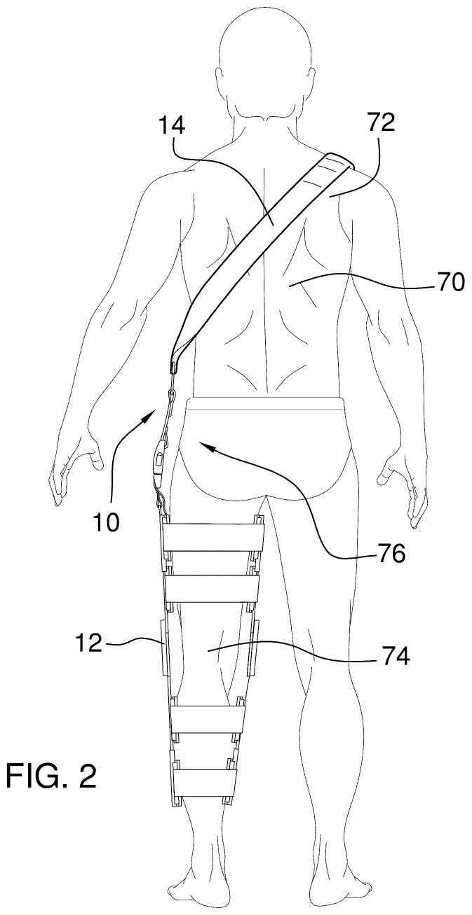

With reference now to the drawings, and in particular to FIGS. 1 through 4 thereof, a new leg brace assembly embodying the principles and concepts of an embodiment of the disclosure and generally designated by the reference numeral 10 will be described.

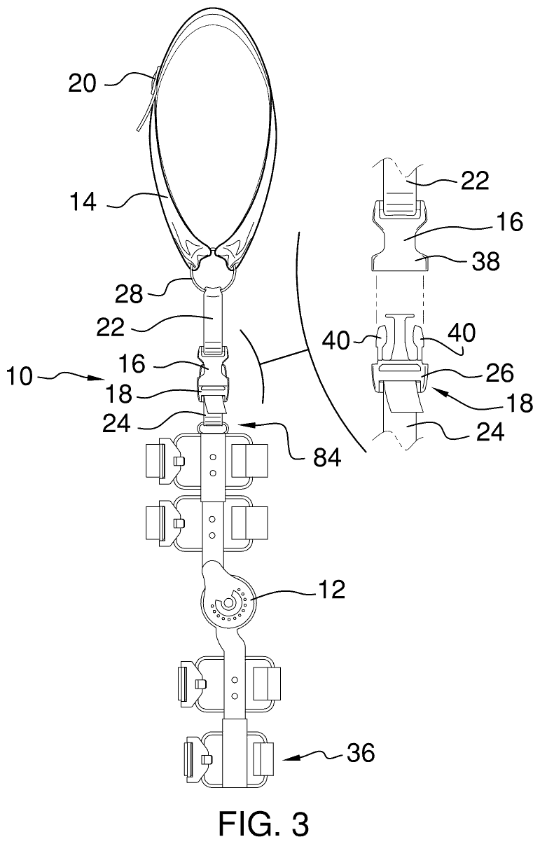

As best illustrated in FIGS. 1 through 4, the leg brace assembly 10 generally comprises a leg brace 12, a strap 14, a first coupler element 16, and a second coupler element 18. In the exemplary embodiment in FIG. 1, the strap 14 is designed to be worn in a loop around a torso 70 and over a shoulder 72 opposite a leg 74 on which the leg brace 12 is worn. As best seen in FIG. 3, the first coupler element 16 is attached to the strap 14. The second coupler element 18 is attached to the leg brace 12. The second coupler element 18 is coupled to the first coupler element 16 to detachably couple the strap 14 to the leg brace 12. As shown in FIG. 1, the first coupler element 16 and the second coupler element 18 extend along a hip 76 of the leg 74 to support the leg brace 12 on the strap 14 in a suspended manner to minimize sliding movement of the leg brace 12 down the leg 74.

The leg brace 12 has a medial side 30, which is positioned on an inner side of the leg 74, and a lateral side 32, which is positioned on an outer side of the leg 74, as is known in the orthopedic arts. The leg brace 12 has a proximal portion 34 and a distal portion 36. As shown in FIGS. 1 and 3, the second coupler element 18 is attached to the lateral side 32 of the leg brace 12 at the proximal portion 34.

In the exemplary embodiment in FIG. 3, the strap 14 includes a buckle assembly 20 designed to adjust the diameter of the loop. The buckle assembly 20 can be any common design, such as a buckle with holes in the strap or a sliding design. In another possible embodiment, the strap 14 could be a single length and could possibly include an elastic material for adjustability. The strap 14 is made of fabric or textile made of natural or synthetic fibers. In another possible embodiment, the strap 14 could be made of hypoallergenic material for wearing against the skin.

The leg brace assembly 10 also includes a first belt 22 and a second belt 24. The first belt 22 connects the first coupler element 16 to the strap 14, and the second belt 24 connects the second coupler element 18 to the leg brace 12.

In the exemplary embodiment in FIG. 3, the second coupler element 18 includes a slider 26, much like are used for shoulder straps for backpacks. The second belt 24 is inserted through the slider 26. The slider 26 is adjustable along the second belt 24 to adjust the distance between the second coupler element 18 and the leg brace 12. In addition, the leg brace assembly 10 also includes a ring 28. The ring 28 connects ends of the strap 14 and the first belt 22 in a Y-shaped orientation. The ring 28 is only one possible type of connection, as the ring 28 could be replaced with a stitched connection between the strap 14 and the first belt 22. In another possible embodiment, the first belt 22 could be omitted and the strap 14 could be directly connected to the first coupler element 16.

In the exemplary embodiment in FIG. 3, the first coupler element 16 is a snap-fit receptacle 38, and the second coupler element 18 includes snap-fit prongs 40 designed to be inserted into the snap-fit receptacle 38. However, this is only one possible type of connection, as the first coupler element 16 and the second coupler element 18 could be connected by other types of connectors, such as snaps, buttons, and zippers.

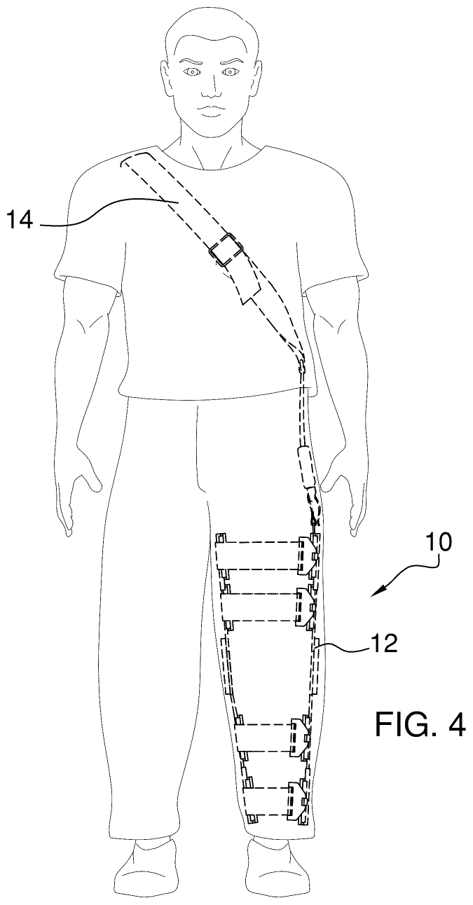

FIGS. 1, 3, and 4 show how the leg brace assembly 10 is used. A person first puts the leg brace 12 on the leg 74 that requires support. The strap 14 is placed about the torso 70 around the torso 70 and over a shoulder 72 opposite the leg 74. The first coupler element 16 and the second coupler element 18 are detachably coupled together, such as by the snap-fit connection. The fit of the strap 14 can then be adjusted by adjusting the diameter of the loop using the buckle assembly 20 to comfortably and snugly fit on the torso 70. In addition, the slider 26 can be adjusted to adjust the distance between the second coupler element 18 and the leg brace 12. The person can now engage in regular physical activities without the leg brace 12 sliding down the leg 74. As shown in FIG. 4, a person can wear the leg brace assembly 10 against his or her body concealed under clothing, or alternatively visible over the clothing.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A leg brace assembly comprising:
a leg brace;
a strap, the strap being configured to be worn in a loop around a torso and over a shoulder opposite a leg on which the leg brace is worn;
a first coupler element, the first coupler element being attached to the strap, the first coupler element being a snap-fit receptacle; and
a second coupler element, the second coupler element being attached to the leg brace, the second coupler element being coupled to the first coupler element to detachably couple the strap to the leg brace, such that the first coupler element and the second coupler element are configured to extend along a hip of the leg to support the leg brace on the strap in a suspended manner to minimize sliding movement of the leg brace down the leg, the second coupler element including snap-fit prongs configured to be inserted into the snap-fit receptacle, said snap fit prongs being manipulatable towards each other to release the snap fit prongs from the snap fit receptacle wherein said second coupler element is releasable from said first coupler element without urging said first coupler element and said second coupler element towards each other.

2. The leg brace assembly of claim 1, wherein:
the leg brace has a medial side and a lateral side;
the leg brace has a proximal portion and a distal portion; and
the second coupler element is attached to the lateral side of the leg brace at the proximal portion.

3. The leg brace assembly of claim 1, wherein the strap includes a buckle assembly configured to adjust the diameter of the loop of the strap.

4. The leg brace assembly of claim 1, further comprising a first belt and a second belt, the first belt connecting the first coupler element to the strap, and the second belt connecting the second coupler element to the leg brace.

5. The leg brace assembly of claim 4, wherein:
the second coupler element includes a slider;
the second belt is inserted through the slider; and
the slider is adjustable along the second belt to adjust the distance between the second coupler element and the leg brace.

6. The leg brace assembly of claim 4, further comprising a ring, the ring connecting ends of the strap and the first belt in a Y-shaped orientation.

7. (canceled)

8. The leg brace assembly of claim 2, wherein the strap includes a buckle assembly configured to adjust the diameter of the loop of the strap.

9. The leg brace assembly of claim 8, further comprising a first belt and a second belt, the first belt connecting the first coupler element to the strap, and the second belt connecting the second coupler element to the leg brace.

10. The leg brace assembly of claim 9, wherein:
the second coupler element includes a slider;
the second belt is inserted through the slider; and
the slider is adjustable along the second belt to adjust the distance between the second coupler element and the leg brace.

11. The leg brace assembly of claim 10, further comprising a ring, the ring connecting ends of the strap and the first belt in a Y-shaped orientation.

12. (canceled)

13. A method of supporting a leg brace to minimize sliding movement of the leg brace down a leg, the method comprising the steps of:
providing a leg brace assembly comprising:
a leg brace;
a strap, the strap being configured to be worn in a loop around a torso and over a shoulder opposite a leg on which the leg brace is worn;
a first coupler element, the first coupler element being attached to the strap, the first coupler element being a snap-fit receptacle; and
a second coupler element, the second coupler element being attached to the leg brace, the second coupler element being coupled to the first coupler element to detachably couple the strap to the leg brace, such that the first coupler element and the second coupler element are configured to extend along a hip of the leg to support the leg brace on the strap in a suspended manner to minimize sliding movement of the leg brace down the leg,, the second coupler element including snap-fit prongs configured to be inserted into the snap-fit receptacle, said snap fit prongs being manipulatable towards each other to release the snap fit prongs from the snap fit receptacle wherein said second coupler element is releasable from said first coupler element without urging said first coupler element and said second coupler element towards each other;
putting the leg brace on a leg;
placing the strap about a torso in a loop around the torso and over a shoulder opposite the leg; and
detachably coupling the first coupler element and the second coupler element.

14. The method of claim 13, wherein the strap includes a buckle assembly configured to adjust the diameter of the loop, and the method further comprising adjusting the diameter of the loop to comfortably and snugly fit on the torso.

15. The method of claim 14, wherein:
the second coupler element includes a slider;
a second belt is inserted through the slider;
the slider is adjustable to along the second belt to adjust the distance between the second coupler element and the leg brace; and
the method further comprises adjusting the slider and thereby adjusting the distance between the second coupler element and the leg brace.

16. The method of claim 15, wherein the method further comprises putting on clothing over the leg brace assembly to the conceal the leg brace assembly.
